(19) United States
(12) Patent Application Publication
(10) Pub. No.: US 2026/0140110 A1
(43) Pub. Date: May 21, 2026

(54) IMMUNOASSAY RAPID DIAGNOSTIC TESTS USING FLUORESCENCE

(71) Applicant: ARIZONA BOARD OF REGENTS ON BEHALF OF THE UNIVERSITY OF ARIZONA, Tucson, AZ (US)

(72) Inventor: Michael WOROBEY, Tucson, AZ (US)

(21) Appl. No.: 19/119,206

(22) PCT Filed: Oct. 11, 2023
(86) PCT No.: PCT/US2023/034887

§ 371 Date: Apr. 8, 2025

Related U.S. Application Data
(60) Provisional application No. 63/379,012, filed on Oct. 11, 2022.

(51) Int. Cl. G01N 33/543; G01N 21/64

(52) U.S. Cl. CPC G01N 33/54388; G01N 21/6428; G01N 2021/6439

(57) ABSTRACT

A visualizer for use with an immunoassay rapid diagnostic test (RDT) with fluorescent probes includes a housing sized to enclose the RDT, an opening in the housing for insertion of the RDT, a viewing window in the housing positioned to align with a result window in the RDT when inserted in the housing, a UV/visible/infrared light source inside the housing for illuminating fluorescent probes in the result window so they may be viewed through the viewing window, and a switch for activating the light source. An RDT incorporating a UV/visible/infrared light source within its housing is also disclosed.

### CROSS-REFERENCE TO RELATED APPLICATIONS

This application claims the benefit of U.S. Provisional Application No. 63/379,012, filed on 11 Oct. 2022, the disclosure of which is incorporated herein by reference in its entirety.

### BACKGROUND

An immunoassay is a biochemical test that detects the presence of a molecule in a solution using an antibody or antigen. Immunoassays are used in a wide variety of rapid diagnostic tests (RDTs) that are single use, inexpensive, and provide results in a matter of minutes. Common formats for RDTs include lateral flow tests and vertical flow tests. RDTs may be used in many industry sectors such as clinical, veterinary, agricultural, food and environmental sectors to confirm the presence or absence of pathogens, biomarkers or contaminants.

Lateral flow test strips have been widely used for detecting the presence of target analytes, such as the SARS-COV-2 antigens, in an individual for diagnostic purposes or in other specimen types like wastewater for detection purposes. Often, these test strips are provided as kits for in-home or point-of-care use where all components of the kit are disposable.

61A is a schematic diagram illustrating principles of a prior art lateral flow test strip. In-home test or point-of care kits may require an individual or a healthcare worker to collect a biological sample, for example by using a swab, immerse the swab in a fluid, then add a few drops of the fluid 102 containing analyte 116 to a sample well containing a sample pad 104 at one end of the lateral flow test strip. Through capillary action, fluid 102 is drawn across the lateral flow test strip to conjugate pad 106.

Conjugate pad 106 stores antibodies 118 or other molecules that bind the analyte, which are conjugated to chemical labels that induce or amplify a signal detectable by the naked eye at sufficiently high concentrations. As sample fluid 102 is drawn into conjugate pad 106, analyte 116 present in sample fluid 102 will bind to the conjugated antibodies and continue to migrate along the test strip.

From conjugate pad 106, the labeled antibodies and analyte 119 flow into a nitrocellulose membrane 108. A row of antibodies 118 corresponding to those on conjugate pad 106 are coated on nitrocellulose membrane 108 at test line 112. If target antigens of the analyte 116 are present in the sample fluid and have bound to the labeled antibodies, those antigens will bind to the monoclonal antibodies at the test line 112. This results in an antibody-antigen-antibody-label that shows as a line visible to the naked eye at the test area, as shown in FIG. 1C. The quality control area (C line 114) is coated with antibodies that bind the labeled antibodies, resulting in a visible C line 114. If the C line 114 does not show any color, it indicates that the result is invalid. Absorbent pad 110 will absorb excess fluid 102.

Prior art in-home test kits may use a variety of labels that are visible to the naked eye, such as colloidal gold nanoparticles, which generate a colorimetric signal and have low sensitivity. However, sample fluids with a small copy number of antigens may not provide enough color in the test area for detection by the naked eye, or inspire confidence in the user that the antigen has been detected. Tests that generate a fluorescent signal can provide much higher sensitivity than colloidal gold colorimetric tests. The fluorescent signal can be generated via excitation of fluorescent labels by ultraviolet (UV) radiation or even visible light, such as near-UV wavelengths in the case of horse radish peroxidase.

Microfluidic devices have been used for detecting the presence of target analytes, such as the SARS-COV-2 antigens, whether in an individual for diagnostic purposes or in other specimen types like wastewater for detection purposes. Microfluidic devices may be used in many industry sectors to confirm the presence or absence of pathogens, biomarkers or contaminants, for example.

FIGS. 1D-1E are schematic diagrams illustrating principles of a prior art microfluidic device 130. Point-of care tests may require the individual or a healthcare worker to collect a biological sample, for example by using a swab, then to immerse the swab in a fluid, then add a few drops of the fluid 102 containing analyte 116 to a loading inlet at one end of a channels 132 of microfluidic device 130. Through capillary action, fluid is drawn across the microfluidic device. In the case of the prior art in FIGS. 1D-1E, the microfluidic device is a paper-based chip with microfluidic channels.

After a period of time, antibody-conjugated fluorescent particles 134 are loaded into each channel 132 and also flows through the paper microfluidic channels passively through capillary action and mixes with sample fluid. If target analytes are present, this mixing leads to particle immunoagglutination 136, which is detected with a fluorescence microscope.

Multiple images must be taken per channel, starting from the particle front (the farthest area that the particles flowed within the channel) and moving the field of view toward the loading inlet, all while refocusing the fluorescence microscope while moving along the channel. The test requires a skilled technician who uses may use a smartphone fluorescence microscope to search for the immunoaggluntinated fluorescent particles. Fluorescent particles cannot be seen by the naked eye. The difficulty in observing fluorescent particles with the naked eye has precluded using them in home tests.

### SUMMARY OF THE EMBODIMENTS

A single use visualizer for use with an immunoassay rapid diagnostic test (RDT) with fluorescent probes includes a housing sized to enclose the RDT, an opening in the housing for insertion of the RDT, a viewing window in the housing positioned to align with a result window in the RDT when inserted in the housing, a UV/visible/infrared light source inside the housing for illuminating fluorescent probes in the result window so they may be viewed through the viewing window, and a switch for activating the UV/visible light source.

An immunoassay rapid diagnostic test (RDT) for detecting respiratory pathogen antigens or other biological or chemical analytes using fluorescent probes includes a housing, a sample well in an upper surface of the housing, a viewing window in the upper surface of the housing, a sample pad positioned to receive the analyte from the sample well, a conjugate pad loaded with the fluorescent probes for binding with the analyte, the conjugate pad positioned adjacent to the sample pad;

a membrane with a result area having immobilized capture probes binding to fluorescent probe-analyte complexes for indicating a presence of analytes, the membrane positioned adjacent to the conjugate pad, a UV/visible/infrared light source inside the housing and positioned to illuminate the result area, and a switch for activating the UV/visible/infrared light source.

A single-use, disposable system for detecting an analyte including a sample collection device for collecting a sample fluid containing the analyte, an immunoassay rapid diagnostic test (RDT) with fluorescent probes for binding to the analyte in the sample fluid and capture probes for displaying a presence of analytes bound to fluorescent probes, and any of the visualizers disclosed herein.

### BRIEF DESCRIPTION OF THE FIGURES

FIGS. 1A-1B illustrate principles of a prior art lateral flow test cartridge.

FIG. 1C illustrates test results of the lateral flow test cartridge of FIGS. 1A-1B.

FIG. 1D-1E illustrate principles of a prior art microfluidic test device.

FIG. 2 is a top view of a lateral flow test cartridge for use with a fluorescence visualizer, in embodiments.

FIG. 3A is a top perspective view of a fluorescence visualizer for a lateral flow test strip, in embodiments.

FIG. 3B is a bottom perspective view of fluorescence visualizer of FIG. 2, in embodiments.

FIG. 4 is a top view of a fluorescence visualizer and test cartridge prior to illumination, in embodiments.

FIG. 5 is a top view of a fluorescence visualizer and test cartridge after illumination, in embodiments.

FIG. 6 is an interior view of the top surface of a fluorescence visualizer, in embodiments.

FIG. 7 is a top view of an LED screen for use with a fluorescence visualizer, in embodiments.

FIGS. 8A and 8B are top views of a multiplex test cartridge and corresponding multiplex visualizer, in embodiments.

FIGS. 9A-9C show lateral flow test cartridges incorporating an ultraviolet or visible light source, in embodiments.

FIG. 10 is a perspective view of a fluorescence visualizer for a microfluidic device, in embodiments.

FIG. 11 is a top view of a vertical flow cartridge for use with a fluorescence visualizer, in embodiments.

FIG. 11A is a cross-sectional view of the vertical flow cartridge of FIG. 11 along lines 11A-11A, in embodiments.

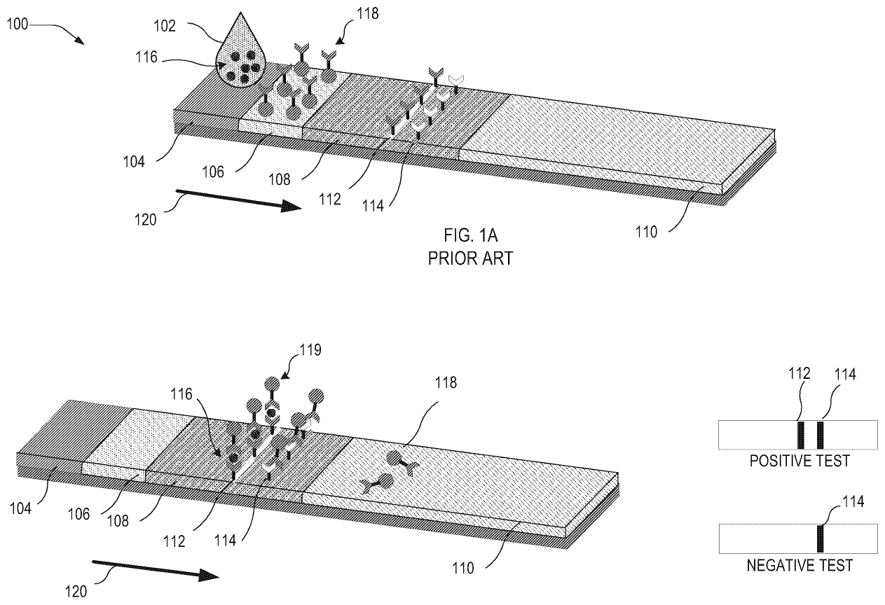

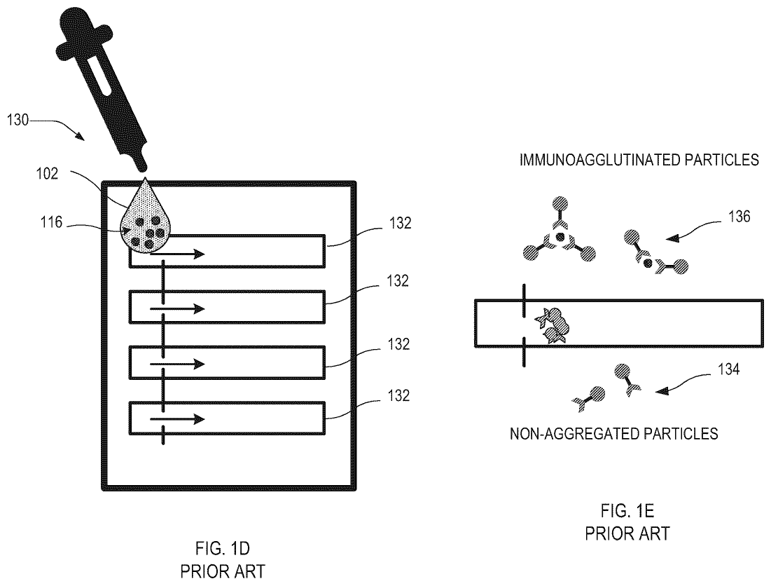

### DETAILED DESCRIPTION OF THE EMBODIMENTS

Rapid diagnostic tests (RDTs) are typically single-use, disposable cartridges that may be used in many industry sectors such as clinical, veterinary, agricultural, food and environmental sectors to confirm the presence or absence of pathogens, biomarkers chemicals or contaminants. They may have a number of form factors, including lateral flow, vertical flow tests and microfluidic devices. Embodiments disclosed herein may be used to detect respiratory viruses such as SARS-COV-2, influenza viruses, and respiratory syncytial viruses (RSV). In addition, blood borne substances such as antibodies or delta-9-tetrahydrocannabinol (THC), various pathogens could also be detected. Embodiments disclosed herein may also be used with environmental contaminants. For purposes of illustration, embodiments will be discussed with regard to the detection of human-borne viruses, but would apply equally to any of the detection of any analyte.

In general, a sample containing an analyte of interest could be acquired from a throat gargle, nasal secretions, saliva, cheek swab, sweat, pus, cerebrospinal fluid, blood, tears, soil, feces, urine, breath, wastewater, or drinking water, for example. Samples may be combined with a liquid solution prior to being used with an RDT.

Lateral flow test strips using fluorescent labels to bind an analyte may have much greater sensitivity to analytes, such as SARS-COV-2 antigens, present in a sample than test strips using colloidal gold nanoparticles, with a limit of detection (LOD) 10 times or greater; however, fluorescent labels are only visible to the naked eye when viewed using an ultraviolet (UV) or near-UV/visible light source of a specific wavelength (for example 405 nm in the case of horse radish peroxidase). As discussed herein, the term “label” may be used interchangeably with the term “probe.” In embodiments, a light source may be any source of electromagnetic radiation source that will induce visibility of selected labels.

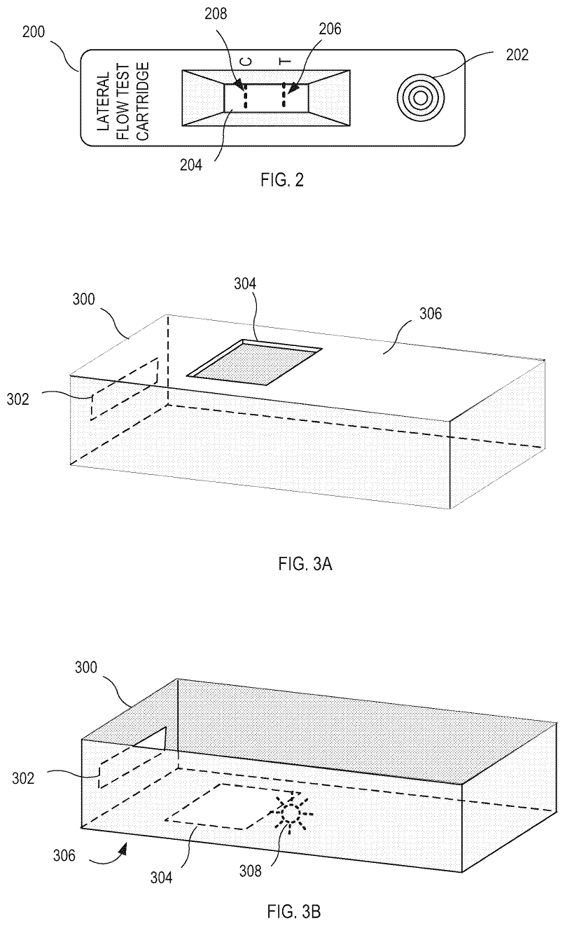

FIG. 2 shows a top view of a representative lateral flow test cartridge 200 for use with the fluorescence visualizer of FIGS. 3A and 3B. Cartridge 200 is shown to illustrate principles of embodiments disclosed herein, which are not limited to the specific cartridge shown.

The lateral flow test cartridge 200 of FIG. 2 may be an in-home test kit purchased by a consumer or a device used at a point-of-care facility. As shown, lateral flow text cartridge 200 is designed to detect one target analyte, such as SARS-COV-2 antigens, for example. However, cartridges may also detect a plurality of analytes, as discussed below. Sample material is collected from a patient in a variety of ways, then loaded into sample well 202. Solid sample material may be combined with a buffer or reagent to provide a sample fluid for use with any of the cartridges described herein. The sample fluid, which potentially contains the target analytes of interest, flows through a conjugate pad (not shown) loaded with fluorescent detection probes which bind with analytes in the sample fluid. Fluid containing detection probe-analyte complexes flows into a porous membrane under results window 204 where detection probe-analyte complexes are captured by capture probes. Capture probes are immobilized in a pattern that indicates a test result under results window 204. The presence or absence of the target analyte in the sample fluid is shown by test line 206 and control line 208. Other arrangements and layouts of test line 206 and control line 208 are contemplated, for example a dot or other symbols. The dimensions of cartridge 200 would be understood by one of ordinary skill in the art.

In embodiments, the conjugate pad of cartridge 200 is loaded with antibodies for detecting the target analyte that are conjugated with fluorescent particles, which are not visible to the naked eye. A fluorescent signal is visible to the naked eye even though it required excitation by specific wavelengths of UV or visible light to be visible. To determine the results of the text, cartridge 200 is inserted into fluorescence visualizer 300 of FIG. 3A.

FIG. 3A shows a top view and FIG. 3B shows a bottom view of fluorescence visualizer 300. In embodiments, visualizer 300 is generally rectangular and sized to be somewhat larger than cartridge 200. Visualizer 300 may be made of any suitable material that that will support the use described herein, such as plastic or cardboard, for example. In embodiments, a housing of visualizer 300 may be convertible between a flat configuration and a configuration having a volume capable of enclosing cartridge 200.

Visualizer 300 includes an opening 302 where cartridge 200 may be inserted. When fully inserted, results window 204 of cartridge 200 is visible through viewing window 304 in a top surface 306 of visualizer 300. Opening 302 is depicted in one end of visualizer 300, however, cartridge 200 may also be inserted from the side. In embodiments, visualizer 300 may also be open on the bottom so that it is placed over cartridge 200 so that results window 204 aligns with viewing window 304, when the cartridge 200 is placed on a flat surface. In further embodiments, visualizer 300 is designed so that the cartridge 200 snaps into a bottom opening of the visualizer 300 and is held in place, so that cartridge 200 and visualizer 300 don't have to be on a flat surface and may be lifted up to the user's eyes by the user.

Once inserted, UV/visible light source 308 inside visualizer 300 is activated to allow viewing of test line 206 and control line 208 in results window 204.

UV/visible light source 308 is positioned inside visualizer 300 in a location that illuminates results window 204. This allows the user to clearly see the “test” and/or “control” lines of results window 204 through viewing window 304. Fluorescence visualizer 300 blocks ambient light and provides a more controlled viewing condition. It also prevents exposure of a user's eyes to UV light. It also provides a fixed position for precise, repeatable readings.

In embodiments, viewing window 304 may simply be an opening in the top surface of fluorescence visualizer 300. In other embodiments, viewing window 304 may also be fitted with a transparent material that would filter out autofluorescence which may contribute to false positives. In other embodiments, viewing window 304 may be made of a material that provides magnification to make visualization of the test and control lines easier. Magnifying test results would assist visually impaired people when reading test results. A viewing window with magnification would also allow a smaller results window 204 in test cartridge 200. Other types of lenses are contemplated.

UV/visible/infrared light source 308 may be a UV/visible light diode of a specific wavelength, for example. As discussed in more detail below, light source 308 may be powered by a battery source inside or external to visualizer 300. Other power sources are contemplated.

Light source 308 may be turned on automatically when a test strip is inserted into the visualizer using a pressure sensitive switch (not shown), for example. Light source 308 may also be turned on with a manual switch on the outside of visualizer 300 that may be activated by a user. In embodiments, visualizer 300 may include additional components as discussed below.

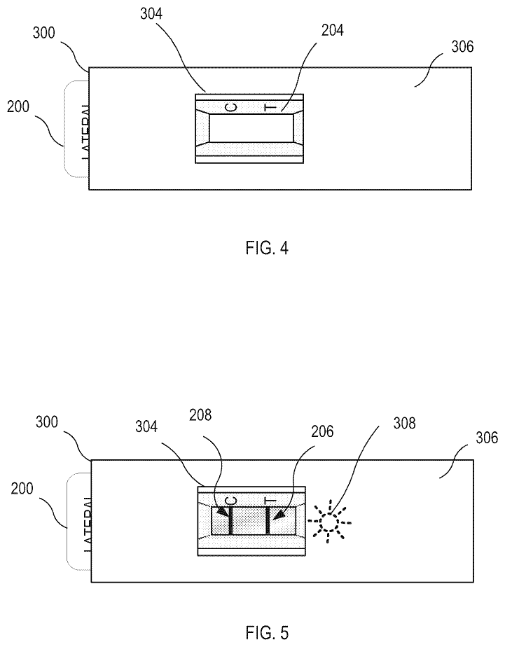

FIG. 4 shows a top view of visualizer 300 and cartridge 200 prior to illumination.

FIG. 5 shows a top view of visualizer 300 and cartridge 200 after illumination. In FIG. 4, no results are visible in results window 204. In FIG. 5, after activation of light source 308 inside visualizer 300, test line 206 and control line 208 are visible. A positive test result is shown in FIG. 5. A negative test result would show only the presence of control line 208 while an invalid test a variety of inconclusive results, such as the presence of test line 206 but not control line 208, or a barely visible version of either of both of test line 206 and control line 208. As shown in FIGS. 3B and 5, light source 308 is positioned above cartridge 200 after insertion. Although a specific location is shown, light source 308 may be positioned in any location on the interior of top surface 306 that provides sufficient illumination to result window 204, including the opposite end or on either side of viewing window 304.

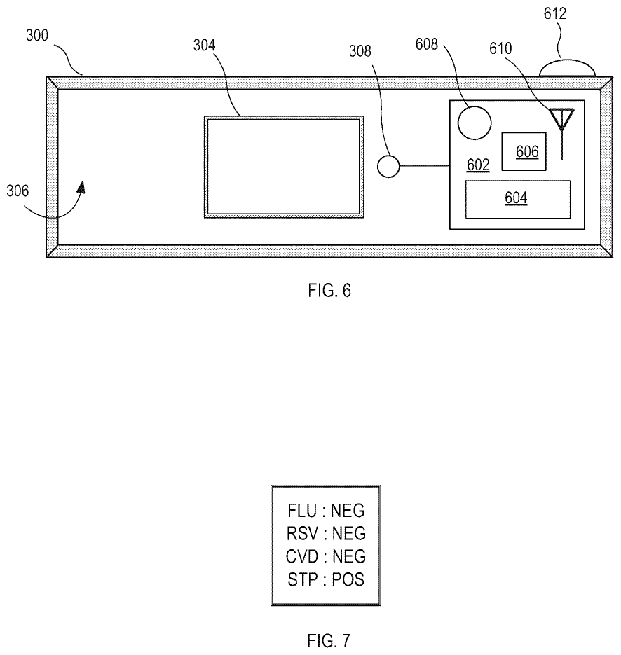

FIG. 6 is an interior view of the top surface of a fluorescence visualizer, in an embodiment. To provide the functions of visualizer 300 as described herein, a variety of components may be provided. A printed circuit board 602 or other substrate may provide support for a processing component 604, a memory 606, a battery 608 and an antenna 610. Sensors may be provided for identifying information from a results window of a cartridge inserted in visualizer 300. Other electronic circuity may be provided. Antenna 610 may communicate with a smartphone or other electronic device using wireless communications capability, for example, a Bluetooth® interface. A visualizer 300 that includes a Bluetooth capability may or may not include viewing window 304. One or more switches 612 may be provided on the outside of visualizer 300 for activation by a user to turn light source 308 on for viewing of test results. A pressure-sensitive switch (not shown) may also be provided on the inside of visualizer 300.

FIG. 7 is a top view of an LED screen for use with a fluorescence visualizer. In embodiments, visualizer 300 including a wireless communication capability may be fully enclosed with no viewing window. In these embodiments, an LED screen 700 may be provided on the outside of visualizer 300 for displaying results to a user. A representative LED screen is shown in FIG. 7, although other information and arrangements of information may be provided. In single test embodiment of FIGS. 2-5 or a multiplex embodiment as described below, an LED/LCD may display an actual analyte name, e.g., FLU A, FLU B, RSV, COVID, or STREP, along with the result for that analyte. An LED/LCD screen may be provided with any of the fluorescence visualizers discussed herein, including those with or without an antenna.

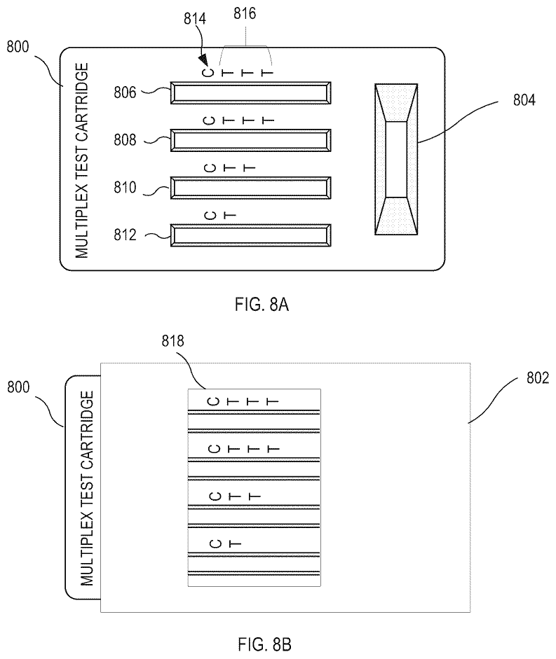

FIGS. 8A and 8B are top views of a multiplex test cartridge 800 and corresponding multiplex visualizer 802, in embodiments.

Multiplex test cartridge 800 includes sample well 804, which is an example of sample well 202. Sample well 804 may be sized to accept a larger quantity of sample fluid than sample well 202 in order to provide enough analyte to perform multiple tests. As shown in FIG. 8A, multiplex test cartridge 800 includes four result windows 806, 808, 810 and 812. In embodiments, each result window 806, 808, 810 and 812 may be associated with an individual test strip within cartridge 800.

In embodiments, each test strip in multiplex test cartridge 800 may also have its own sample well 804. Or test strips may share sample wells such that there is more than one sample well but fewer than the total number of test strips. As shown in FIG. 8A, multiplex test cartridge 800 includes four result windows 806, 808, 810 and 812 each having a control line 814 and up to three test lines 816.

Multiplex visualizer 802 is an example of fluorescence visualizer 300. Viewing window 818 may be wider to accommodate multiple results windows 806, 808, 810 and 812. In embodiments, each result window may be illuminated by a single UV/visible light source. Alternatively, each result window may be associated with an individual light source. Any number of UV/visible light sources in any position with multiplex visualizer may be used as necessary to adequately illuminate result windows 806, 808, 810 and 812.

The multiplex test cartridge 800 and multiples visualizer 802 of FIGS. 8A and 8B would allow for visualization, without instrumentation, of the presence or absence of multiple analytes, for example multiple respiratory pathogen antigens from a single sample (e.g., respiratory viruses such as H3N2 influenza virus, H1N1 influenza virus, influenza B virus, SARS-COV-2, RSV-A, RSV-B, or respiratory bacteria such as group A streptococcus and Bordetella pertussis. Further, the use of one or more diodes to illuminate the samples facilitates taking a photo with a smartphone of the illuminated results, which may perform a number of tasks with the captured image, including identifying information with the image using image processing algorithms, sending it to health care providers or public health officials, or saving it, for example.

In embodiments, multiplex test cartridge 800 may be capable of simultaneously testing for as many as 18 different analytes, such as 18 different respiratory pathogens, including not only viruses but also bacteria like group A strep. This could be accomplished by using a different types of nitrocellulose paper for material tests, with all test strips could be fed from the same sample pad and the user putting all necessary drops in one sample well 804.

Although FIG. 8A shows four result windows 806, 808, 810 and 812 (and underlying test strips), each with a control line 814 and up to three test lines 816, any number of test strips and lines per test strips could be used. A larger number of test strips and lines could be used in conjunction with a magnifying lens in viewing window 818 of multiplex visualizer 802 as discussed above, wherein test areas could be miniaturized but still visible with the naked eye. Multiplex visualizer 802 may include the internal electronic circuitry of FIG. 6 and the LED screen of FIG. 7, as described above.

For the detection of analytes including pathogen antigens, the use of fluorescent probes rather than colorimetric probes, such as colloidal gold, offers the opportunity to generate much higher sensitivity tests, with limits of detection (LOD) a tenth the concentration or less compared colloidal gold. This is because some fluorescent probes, when excited by specific wavelengths of UV, visible or infrared radiation can generate more intense signals than the colorimetric signals of colloidal gold. Selection of fluorescent probes for binding to sample analytes may be done according to techniques known in the art. Of particular importance is a large Stokes shift, the difference between the emission wavelength and the excitation wavelength of the fluorescent probe. Fluorophores that can exhibit large Stokes shifts include quantum dots (QDs), and monoclonal antibodies that bind to analytes can be conjugated directly to QDs. Embodiments disclosed herein may be used with colorimetric, chemiluminescent, or fluorescent probes.

Prior art fluorescent probes may also comprise monoclonal antibodies conjugated to fluorescent QD-nanoparticle complexes that provide a stronger fluorescent band in positive samples and therefore higher sensitivity. Polystyrene microbeads infused with lanthanide chelates, such europium chelate, which exhibits a large Stokes shift (280 nm) and a very long lifetime, are also available (e.g., Fluoro-Max™ Fluorescent Carboxylate-Modified Particles) and can generate high sensitivity tests.

Embodiments disclosed thus far encompass a single or multiplex test cartridge where antibodies for a target analyte are conjugated with fluorescent particles. These test cartridges are used with a separate visualizer, or reader, to display test results. A test cartridge and visualizer may be incorporated into an in-home or point-of-care test kit. The devices disclosed herein could be produced at a cost that is feasible for a disposable use and would allow in-home tests and point of care tests for SARS-COV-2 and other pathogens to be much more sensitive without losing any of the convenience. In embodiments, a kit could be provided with a plurality of test cartridges for one visualizer.

In a further embodiment, a test cartridge and a visualizer may be incorporated into a single device.

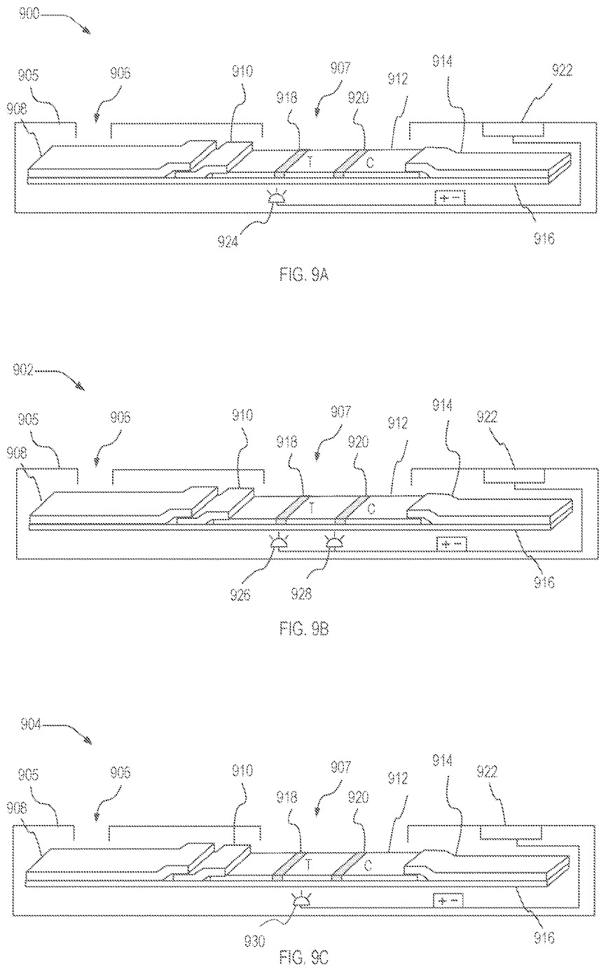

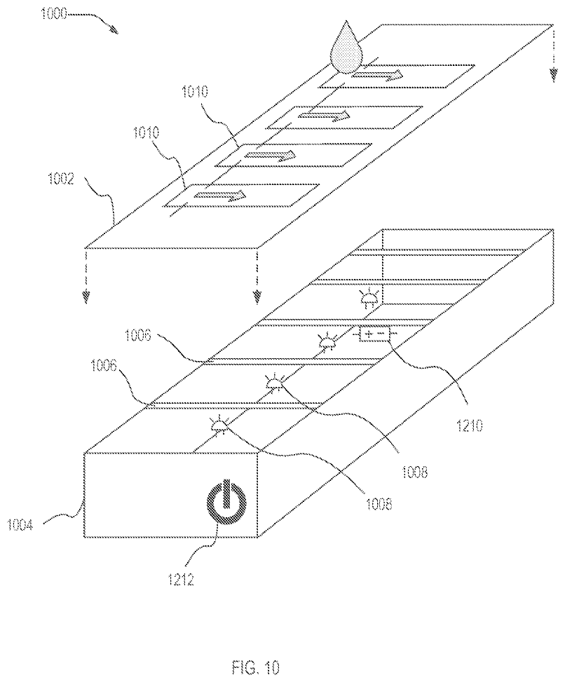

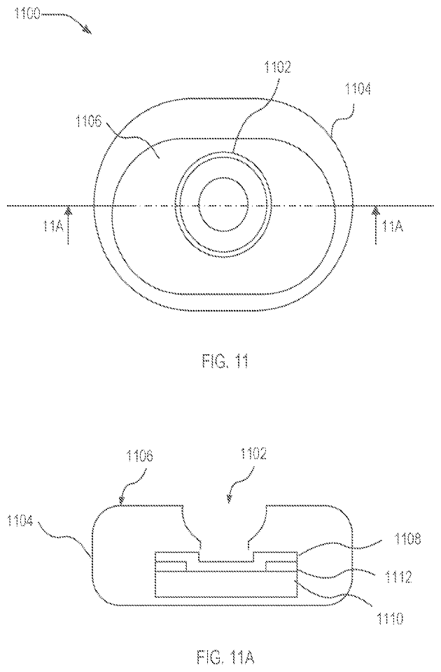

FIGS. 9A-9C show test cartridges 900, 902 and 904 incorporating an electromagnetic radiation source within the body of the test cartridge. In embodiments, a lateral flow test cartridge 900 incorporates a UV/visible light source 924 within housing 905, as shown in FIG. 9A. Housing 905 includes a sample well 906 for receiving a sample fluid potentially containing a target analyte. Sample well 906 is an example of sample well 206. Sample pad 908, conjugate pad 910, nitrocellulose membrane 912 and absorbent pad 914 are positioned along the length of housing 1102 and may be supported by a backing 916 that may be transparent or translucent. Sample pad 908 is positioned at one end of housing 905 below sample well 906 for receiving the sample fluid containing a target analyte. Conjugate pad 910 is loaded with antibodies for detecting the target analyte that are conjugated with fluorescent particles. Capillary action causes the sample fluid to move from sample pad 908 through conjugate pad 910, where any analyte present in the sample fluid binds to the conjugated antibodies, to nitrocellulose membrane 912, and then to absorbent pad 914.

If target antigens are present in the sample fluid and have bound to the labeled antibodies from conjugate pad 910, those antigens will bind to the corresponding antibodies at the test line 918. This results in an antibody-antigen-antibody-label that shows as a line visible to UV light. Conjugated antibodies will also bind to antibodies at the control line 920. Switch 922 may be used to activate UV/visible light source 924 underneath the T line. In embodiments, UV/visible light source may be activated using a timer or other mechanism. Because backing 916 is transparent, when UV/visible light source 924 is activated, the result area and the T line will be visible through result window 907.

FIG. 9A shows an embodiment of a test cassette with one UV/visible light source positioned under the test line. In this embodiment, the control line does not require UV illumination because it is based on a colorimetric indicator like gold nanoparticles, for example.

FIGS. 9B and 9C show other embodiments of test cartridge 900. Like numerals in different drawings represent like or similar elements unless otherwise indicated. FIG. 9B shows a test cartridge 902 with two light sources 926 and 928 for separate illumination of the test and control lines. This may be used in embodiments where both test line 918 and control line 920 use fluorescent probes. FIG. 9C shows test cartridge 904 where both the test and control lines use fluorescent probes but are both illuminated with a single, central light source 930. Other numbers and positions of one or more UV/visible light sources are contemplated, both under backing 916 and above it as discussed above with reference to FIGS. 2-5.

In embodiments, the material of backing 916 below the results area may be a band pass filter material (like a UV filter on a camera lens) that lets through only the correct wavelengths to excite the fluorophore or other reporter probes but blocks other wavelengths. In this case, a diode with the exact wavelength required for fluorophore labels in conjugate pad 910 would not be necessary. In addition, because a band pass filter material appears almost black to the naked eye, it would provide a better contrast for visualizing fluorescence as compared to clear material.

Any of the features discussed herein may be incorporated into lateral flow test cartridges 900, 902 and 904, including an LED display, internal sensors, electronic circuitry and Bluetooth connectivity. Further, result window 907 may be a transparent material that blocks potentially harmful UV radiation or other light spectrum from damaging a viewer's eyes. In embodiments, a transparent material used for result window 907 may be also provide magnification in addition to or instead of blocking UV radiation. Further, embodiments with internal sensors and electronic circuitry may not include a result window 907 and may instead use a sensor to detect the test and control lines, then provide the result to a user digitally.

FIG. 10 shows a fluorescence visualizer 1000 for a microfluidic device 1002. Base unit 1004 may be an enclosed box with a clear top surface. Although base unit 1004 is generally rectangular as depicted, any shape may be used. Further, base unit 1004 may not be the same size as microfluidic device 1002, as long as it is big enough to provide illumination to the results area of microfluidic device 1002. Base unit 1004 may include one or more cross braces 1006 to provide support for microfluidic device 1002.

One or more UV/visible light sources 1008 may be positioned inside base unit 1004, either on cross braces 1006 as shown in FIG. 10 or at other locations. Light sources 1008 may be positioned at any location where sufficient illumination is provided to read test results in microfluidic device 1002. Microfluidic device 1002 including one or a plurality of test areas 1010 may be placed on top of base unit 1004. Light sources 1008 may be manually activated using switch 1212. Light sources 1008 may also be activated by a pressure sensitive switch activated when microfluidic device 1002 makes contact with the top of base unit 1004. The visualizer of FIG. 10 allows a user to see UV-excitation fluorescent labels in test areas 1010 with the naked eye. In embodiments, a smartphone may be used to take pictures of test areas 1010 when illuminated for analyses.

Principles of fluorescence disclosed herein may also be applied to a vertical flow test cartridge 1100, such as that shown in FIGS. 11-11A.

FIG. 11 is a top view of a representative vertical flow immunoassay RDT. Although a physical shape and arrangement is shown in FIG. 11, other shapes and arrangements are within the scope of this disclosure. FIG. 11A is a cross-sectional view of vertical flow cartridge 1100.

FIGS. 11 and 11A are best viewed together in the following description. Vertical flow cartridge 1100 may be used with a sample fluid containing an analyte of interest as described above. In embodiments, a vertical flow RDT operate similarly to lateral flow tests but use gravity to move sample and conjugates through a membrane at the bottom of a large sample well.

A housing 1104 of vertical flow cartridge 1100 is made from plastic or a similar material. A representative internal structure of vertical flow cartridge 1100 will be described, but other structures are possible as long as it includes fluorescent detection probes for identifying an analyte. Sample well 1102 is located in a central region of the top surface 1106 of housing 1104. Porous membrane 1108 is positioned to receive a sample fluid from sample well 1102. Absorbent pad 1110 is positioned under porous membrane 1108 to absorb extra sample fluid. Support structure 1112 may be an annular ring of a non-porous material to confine sample fluid in the central area of porous membrane 1108.

The vertical flow cartridge of FIG. 11 may be used with a visualizer similar to visualizer 300 of FIGS. 3A and 3B. In embodiments, any of the visualizers disclosed herein are sized to enclose an RDT to reduce or block out ambient light. In embodiments, dimensions of a visualizer are approximately 5 to 50% larger than the dimensions of the RDT. A visualizer may have an elongated rectangular prism shape as shown in FIG. 3A, or an ovoid shape to enclose the vertical flow cartridge of FIG. 11. Other shapes are contemplated. An RDT may be inserted through an opening in a visualizer. In embodiments, a visualizer as disclosed herein may be open on a bottom surface opposite the top surface so that it can be placed on top of a RDT when it is resting on a flat surface. In embodiments, a visualizer may be designed to attach to an RDT with a snap fit.

In further embodiments, vertical flow cartridge 1100 may be an example of any of test cartridges 900, 902 or 904. Housing 1104 may incorporate a UV/visible light source positioned to illuminate porous membrane 1108. The UV visible light source may be positioned under or above porous membrane 1108, for excitation of fluorophores.

Changes may be made in the above methods and systems without departing from the scope hereof. For example, As disclosed herein, test results are shown as lines across the width of a test strip in a result window however, other symbols such as “+” for positive, “-” for negative or “!” for invalid, could be used. Letters such as “POS,” “NEG,” or “INV” could also be used. Additional markings may be provided on any of the embodiments discussed, such as pointers or labels for result areas, instructions for use, a reference spot, a trade mark, a serial number, a bar code, a QR code, RFID-tag etc. (not shown).

It should thus be noted that the matter contained in the above description or shown in the accompanying drawings should be interpreted as illustrative and not in a limiting sense. Herein, and unless otherwise indicated: (a) the adjective “exemplary” means serving as an example, instance, or illustration, and (b) the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

What is claimed is:

1. A single use visualizer for use with an immunoassay rapid diagnostic test (RDT) with fluorescent probes, comprising:
a housing sized to enclose the RDT;
an opening in the housing for insertion of the RDT;
a viewing window in the housing positioned to align with a result window in the RDT when inserted in the housing;
a UV/visible/infrared light source inside the housing for illuminating fluorescent probes in the result window so they may be viewed through the viewing window; and
a switch for activating the UV/visible/infrared light source.

2. The visualizer of claim 1, wherein the results induced or amplified via the light source are at least one of colorimetric, chemiluminescent, or fluorescent.

3. The visualizer of claim 1, wherein the viewing window further comprises a filter for blocking light spectrum that is harmful to eyesight.

4. The visualizer of claim 1, wherein the viewing window further comprises a magnifying material.

5. The visualizer of claim 1, wherein the RDT is a lateral test flow cartridge, a vertical test flow cartridge, or a microfluidic device.

6. The visualizer of claim 1, wherein the housing is made of plastic.

7. The visualizer of claim 1, wherein the housing is made of cardboard that is convertible between a flat configuration and a configuration having a volume.

8. An immunoassay rapid diagnostic test (RDT) for detecting respiratory pathogen antigens or other biological or chemical analytes using fluorescent probes, comprising:
a housing;
a sample well in an upper surface of the housing;
a viewing window in the upper surface of the housing;
a sample pad positioned to receive the analyte from the sample well;
a conjugate pad loaded with the fluorescent probes for binding with the analyte, the conjugate pad positioned adjacent to the sample pad;
a membrane with a result area having immobilized capture probes binding to fluorescent probe-analyte complexes for indicating a presence of analytes, the membrane positioned adjacent to the conjugate pad;
a UV/visible/infrared light source inside the housing and positioned to illuminate the result area; and
a switch for activating the UV/visible/infrared light source.

9. The RDT of claim 8, wherein the UV/visible/infrared light source is positioned above the results area.

10. The RDT of claim 8, wherein the UV/visible/infrared light source is positioned below the results area.

11. The RDT of claim 8, wherein the viewing window further comprises a magnifying material or a filter for blocking some light spectrums.

12. A single-use, disposable system for detecting an analyte, comprising:
a sample collection device for collecting a sample fluid containing the analyte;
an immunoassay rapid diagnostic test (RDT) with fluorescent probes for binding to the analyte in the sample fluid and capture probes for displaying a presence of analytes bound to fluorescent probes; and
the visualizer of claim 1.

13. The system of claim 12, wherein the RDT comprises a lateral flow test cartridge.

14. The system of claim 12, wherein the RDT comprises a vertical flow test cartridge.

15. The system of claim 12, wherein the RDT is a microfluidic device.

16. The system of claim 12, wherein the RDT further comprises fluorescent probes for detecting a plurality of analytes.

17. The system of claim 16, wherein the RDT further comprises capture probes for displaying the presence of the plurality of analytes.

18. A single-use, disposable system for detecting an analyte, comprising:
a sample collection device for collecting a sample material containing the analyte; and
the immunoassay rapid diagnostic test (RDT) of claim 8.

19. The system of claim 18, wherein the RDT further comprises fluorescent probes for detecting a plurality of analytes.

20. The system of claim 19, wherein the RDT further comprises capture probes for displaying the presence of the plurality of analytes.
